(19) United States
(12) Patent Application Publication
(10) Pub. No.: US 2009/0110733 A1
(43) Pub. Date: Apr. 30, 2009

(54) DELIVERY MEANS

(75) Inventors: Nicholas John Crowther, Bradford West Yorkshire (GB); Donald Eagland, Huddersfield West Yorkshire (GB); Stephen Britland, Bradford (GB)

Correspondence Address: MICHAEL BEST & FRIEDRICH LLP, 100 E WISCONSIN AVENUE, Suite 3300, MILWAUKEE, WI, 53202, US

(73) Assignee: AGT SCIENCES LIMITED, Bradford (GB)

(21) Appl. No.: 12/300,761

(22) PCT Filed: May 16, 2007
(86) PCT No.: PCT/GB07/01804

§ 371 Date: Nov. 13, 2008

(30) Foreign Application Priority Data
May 17, 2006 (GB) 0609778.6

(51) Int. Cl. A61K 9/00; A61K 35/64; A61P 17/02

(52) U.S. Cl. 424/486; 424/484; 424/538

(57) ABSTRACT

A delivery means for delivering a deliverable material to a wound bed comprises a hydrogel (preferably an optionally cross-linked polyvinylalcohol) and a deliverable material comprising: (i) a decomposable material which in the absence of said hydrogel decomposes and/or is permanently denatured if sterilised using heat, electron beam radiation or gamma radiation; (ii) a protein, protein fragment, peptide or amino acid; or (iii) a secretion or excretion from the organism Lucilia sericata or Drosophila melanogaster.

This invention relates to a delivery means and particularly, although not exclusively, relates to a delivery means for delivering a deliverable material to a locus especially to a wound bed. Preferred embodiments relate to delivery means in the form of wound care devices for delivery of secretions or excretions from the organism Lucilia Sericata to a wound bed.

It is known, for example from WO01/31033, WO03/075654 and WO03/043669, to treat wounds using proteins and/or secretions and/or excretions of the larval form of the green bottle fly, Lucilia Sericata. Such disclosures describe the incorporation of the proteins and/or secretions and/or excretions into dressings, although no detail is provided on the nature of the dressings or how the deliverable materials are incorporated into dressings. In fact, incorporation of proteins or other natural extracts from living organisms is not trivial because such materials tend to be relatively unstable and susceptible to decomposition or denaturisation under relatively mild conditions of temperature or ionising radiation. Consequentially, it is difficult to sterilise dressings or other delivery means which may be used to deliver such natural extracts because sterilisation involves use of conditions (e.g. greater than 60° C. and/or exposure to gamma radiation of greater than 40 kV) which tend to decompose and/or denature the extracts.

It is an object of the present invention to address the aforementioned problems. Particularly, although not exclusively, it is an object of the invention to provide a delivery means incorporating a relatively unstable material which is normally susceptible to decomposition or denaturisation but which, in the delivery means, can be sterilised under appropriate sterilisation conditions.

According to a first aspect of the invention, there is provided a delivery means for delivering a deliverable material for example to a wound bed, said delivery means comprising a hydrogel and said deliverable material comprising:

(i) a decomposable material which in the absence of said hydrogel decomposes and/or is permanently denatured if sterilised using heat, electron beam radiation or gamma radiation;

(ii) a protein, protein fragment, peptide or amino acid; or

(iii) a secretion or excretion from the organism Lucilia sericata or Drosophila melanogaster.

Advantageously, the hydrogel is found to stabilise and/or protect said deliverable material against decomposition and/or denaturation and consequently the hydrogel containing the deliverable material can be sterilised using conditions which might otherwise decompose or denature the deliverable material such as relatively high temperature or ionising radiation, such as gamma or electron beam radiation.

Referring to (i), the decomposable material may be such that in water (e.g. in a solvent which consists essentially of water) in the absence of said hydrogel, it decomposes and/or is permanently denatured if sterilised as described. For example, said decomposable material may, in the absence of said hydrogel, decompose and/or be permanently denatured if subjected to: a temperature of greater than 100° C. (e.g. in steam sterilisation at greater than 120° C. for at least 20 minutes); gamma radiation of at least 40 kV for at least 1 minute; or electron beam radiation of at least 18 kV for at least 1 minute.

Said hydrogel preferably comprises an optionally derivatised, for example cross-linked, hydrophilic polymer. The hydrophilic polymer may include relatively hydrophilic regions and relatively hydrophobic regions.

Said hydrophilic polymer may comprise optionally-derivatised e.g. cross-linked water soluble gums, for example gum arabic, karaya gum, tragacanth gum, ghatti gum, guar gum; soybean derivatives, for example locust bean gum, tamarind gum; water soluble biopolymers, for example dextran, xanthan gum; water soluble proteins, for example gelatin type materials, carrageenan, agar and alginates, animal derivatives, casein, pectin; starch and starch derivatives, for example starch, modified starch, starch derivatives; cellulose derivatives, for example methyl cellulose, carboxymethyl cellulose, hydroxyethyl cellulose, hydroxypropyl cellulose; polyvinyls and maleic anhydride copolymers, for example polyvinyl alcohol, polyvinyl pyrrolidone; miscellaneous water soluble polyvinyls, for example maleic anhydride copolymers; polyacrylates and related systems, for example polyacrylates, polyacrylamides; polyimines and related systems, for example polyethylene oxides, polyethyleneimines, polyethylene glycols; surface active water soluble polymers, for example lignosulfonates and related materials, lignites, tannins.

Preferred examples of suitable hydrophilic polymers include polymethacrylic acid polymers; polyimides; polyvinylalcohol and copolymers of the aforesaid.

Said hydrophilic polymer preferably includes a carbon atom containing backbone. The carbon atoms are preferably linked together by C—C single bonds. The backbone preferably includes no other types of atoms.

Said hydrophilic polymer preferably includes carbonyl moieties. Such moieties may be included in groups pendent from a backbone of the polymer. Said carbonyl moieties may be components of carboxylic acids or carboxylic acid derivates. Preferably carbonyl moieties are components of ester functional groups, for example groups —OCO—R¹⁰ wherein R¹⁰ represents an optionally-substituted alkyl or alkenyl moiety, especially a C₁₋₄ alkyl or alkenyl moiety. R¹⁰ is preferably an unsubstituted alkyl moiety especially a methyl group. Thus, said hydrophilic polymer preferably includes acetate moieties.

Said hydrophilic polymer preferably includes hydroxyl groups which are suitably pendent from a backbone of the polymer. Preferably hydroxyl groups are bonded directly to the backbone, preferably carbon atoms thereof. Preferred hydroxy groups comprise alcohol functional groups.

Said hydrophilic polymer preferably includes both carbonyl moieties as described and hydroxyl moieties as described, wherein suitably the carbonyl moieties and hydroxyl moieties are present in separate functional groups pendent from the polymer backbone.

Suitably at least 50 mole %, preferably at least 75 mole %, more preferably at least 95 mole %, especially about 100 mole % of said hydrophilic polymer is made up of repeat units which include functional groups which include carbonyl moieties (preferably as part of carboxylic acid or carboxylic acid derivative functional groups) or hydroxyl (especially alcohol) moieties. Suitably, the sum of the mole % of carbonyl containing functional group (e.g. carboxylic acid or carboxylic acid derivative functional groups) and hydroxyl (especially alcohol) functional groups in said hydrophilic polymer is at least 70 mole %, preferably at least 90 mole %, more preferably at least 95 mole %, especially about 100 mole %. Thus, in a preferred embodiment, an hydrophilic polymer material which includes the aforementioned functional groups is not a copolymer which includes other types of functional groups.

Said hydrophilic polymer preferably comprises a polyvinyl polymer. Suitably the sum of the mole % of vinyl moieties in said polymer is at least 70 mole %, preferably at least 90 mole %, more preferably at least 95 mole %, especially about 100 mole %.

The most preferred hydrogel comprises an optionally-derivatised, for example cross-linked, polyvinylalcohol. Preferred polyvinylalcohols include hydroxyl functional groups which are relatively hydrophilic and acetate functional groups which are relatively hydrophobic.

Said hydrogel preferably comprises an optionally-derivatised polyvinylalcohol which suitably consists essentially of vinylalcohol and vinyl acetate functional groups. Suitably, the polyvinylalcohol is hydrolyzed to an extent of less than 100 mole %, preferably less than 95 mole %. It may be hydrolysed to an extent of at least 10 mole %, preferably at least 25 mole %, more preferably at least 50 mole %, especially at least 60 mole %. Suitably, in said polyvinylalcohol, the ratio of the mole % of vinylalcohol moieties to vinylacetate moieties is at least 0.5, preferably at least 1, more preferably at least 3. The ratio may be less than 10, preferably less than 8.

Preferred polyvinylalcohols have a viscosity (measured on a 4% aqueous solution at 20° C.) of at least 2 mPa·s, preferably at least 4 mPa·s. The viscosity may be less than 100 mPa·s, preferably less than 75 mPa·s,

Said hydrophilic polymer of said hydrogel is preferably cross-linked by a cross-linking means

A preferred cross-linking means comprises a chemical cross-linking material. Such a material is preferably a polyfunctional compound having at least two functional groups capable of reacting with functional groups of said hydrophilic polymer. Preferably, said cross-linking material includes one or more of carbonyl, carboxyl, hydroxy, epoxy, halogen or amino functional groups which are capable of reacting with groups present along the polymer backbone or in the polymer structure of the hydrophilic polymer. Preferred cross-linking materials include at least two aldehyde groups. Thus, in a preferred embodiment, said hydrogel includes a material formed by cross-linking polyvinylalcohol using a material having at least two aldehyde groups. Thus, said hydrogel may include a moiety of formula I.

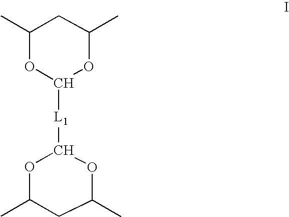

wherein L¹ is a residue of said cross-linking material.

Said cross-linking material preferably comprises a second polymeric material. Said second polymeric material preferably includes a repeat unit of formula

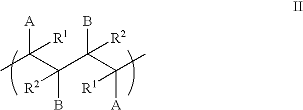

wherein A and B are the same or different, are selected from optionally-substituted aromatic and heteroaromatic groups and at least one comprises a relatively polar atom or group and R¹ and R² independently comprise relatively non-polar atoms or groups.

A and/or B could be multi-cyclic aromatic or heteroaromatic groups. Preferably, A and B are independently selected from optionally-substituted five or more preferably six-membered aromatic and heteroaromatic groups. Preferred heteroatoms of said heteroaromatic groups include nitrogen, oxygen and sulphur atoms of which oxygen and especially nitrogen, are preferred. Preferred heteroaromatic groups include only one heteroatom. Preferably, a or said heteroatom is positioned furthest away from the position of attachment of the heteroaromatic group to the polymer backbone. For example, where the heteroaromatic group comprises a six-membered ring, the heteroatom is preferably provided at the 4-position relative to the position of the bond of the ring with the polymeric backbone.

Preferably, A and B represent different groups. Preferably, one of A or B represents an optionally-substituted aromatic group and the other one represents an optionally-substituted heteroaromatic group. Preferably A represents an optionally-substituted aromatic group and B represents an optionally-substituted heteroaromatic group especially one including a nitrogen heteroatom such as a pyridinyl group.

Unless otherwise stated, optionally-substituted groups described herein, for example groups A and B, may be substituted by halogen atoms, and optionally substituted alkyl, acyl, acetal, hemiacetal, acetalalkyloxy, hemiacetalalkyloxy, nitro, cyano, alkoxy, hydroxy, amino, alkylamino, sulphinyl, alkylsulphinyl, sulphonyl, alkylsulphonyl, sulphonate, amido, alkylamido, alkylcarbonyl, alkoxycarbonyl, halocarbonyl and haloalkyl groups. Preferably, up to 3, more preferably up to 1 optional substituents may be provided on an optionally substituted group.

Unless otherwise stated, an alkyl group may have up to 10, preferably up to 6, more preferably up to 4 carbon atoms, with methyl and ethyl groups being especially preferred.

Preferably, A and B each represent polar atoms or group—that is, there is preferably some charge separation in groups A and B and/or groups A and B do not include carbon and hydrogen atoms only.

Preferably, at least one of A or B includes a functional group which can undergo a condensation reaction, for example on reaction with said hydrophilic polymer.

Preferably, A includes a said functional group which can undergo a condensation reaction.

Preferably, one of groups A and B includes an optional substituent which includes a carbonyl or acetal group with a formyl group being especially preferred. The other one of groups A and B may include an optional substituent which is an alkyl group, with an optionally substituted, preferably unsubstituted, C₁₋₄ alkyl group, for example a methyl group, being especially preferred.

Preferably, A represents a group, for example an aromatic group, especially a phenyl group, substituted (preferably at the 4-position relative to polymeric backbone when A represents an optionally-substituted phenyl group) by a formyl group or a group of general formula

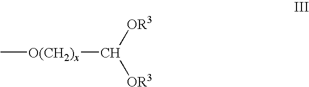

where x is an integer from 1 to 6 and each R³ is independently an alkyl or phenyl group or together form an alkalene group.

Preferably, B represents an optionally-substituted heteroaromatic group, especially a nitrogen-containing heteroaromatic group, substituted on the heteroatom with a hydrogen atom or an alkyl or aralkyl group. More preferably, B represents a group of general formula

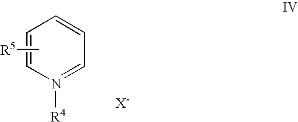

wherein R⁴ represents a hydrogen atom or an alkyl or aralkyl group, R⁵ represents a hydrogen atom or an alkyl group and X⁻ represents a strongly acidic ion. It may be an organic, for example alkyl, sulphate such a methylsulphate.

Preferably, R¹ and R² are independently selected from a hydrogen atom or an optionally-substituted, preferably unsubstituted, alkyl group. Preferably, R¹ and R² represent the same atom or group. Preferably, R¹ and R² represent a hydrogen atom.

Preferred second polymeric materials may be prepared from any of the following monomers by the method described in WO98/12239 and the content of the aforementioned document is incorporated herein by reference:

α-(p-formylstyryl)-pyridinium, γ-(p-formylstyryl)-pyridinium, α-(m-formylstyryl)-pyridinium, N-methyl-α-(p-formylstyryl)-pyridinium, N-methyl-β-(p-formylstyryl)-pyridinium, N-methyl-α-(m-formylstyryl)-pyridinium, N-methyl-α-(o-formylstyryl)-pyridinium, N-ethyl-α-(p-formylstyryl)-pyridinium, N-(2-hydroxyethyl)-α-(p-formylstyryl)-pyridinium, N-(2-hydroxyethyl)-γ-(p-formylstyryl)-pyridinium, N-allyl-α-(p-formylstyryl)-pyridinium, N-methyl-γ-(p-formylstyryl)-pyridinium, N-methyl-γ-(m-formylstyryl)-pyridinium, N-benzyl-α-(p-formylstyryl)-pyridinium, N-benzyl-γ-(p-formylstyryl)-pyridinium and N-carbamoylmethyl-γ-(p-formylstyryl)-pyridinium. These quaternary salts may be used in the form of hydrochlorides, hydrobromides, hydroiodides, perchlorates, tetrafluoroborates, methosulfates, phosphates, sulfates, methane-sulfonates and p-toluene-sulfonates.

Also, the monomer compounds may be styrylpyridinium salts possessing an acetal group, including the following:

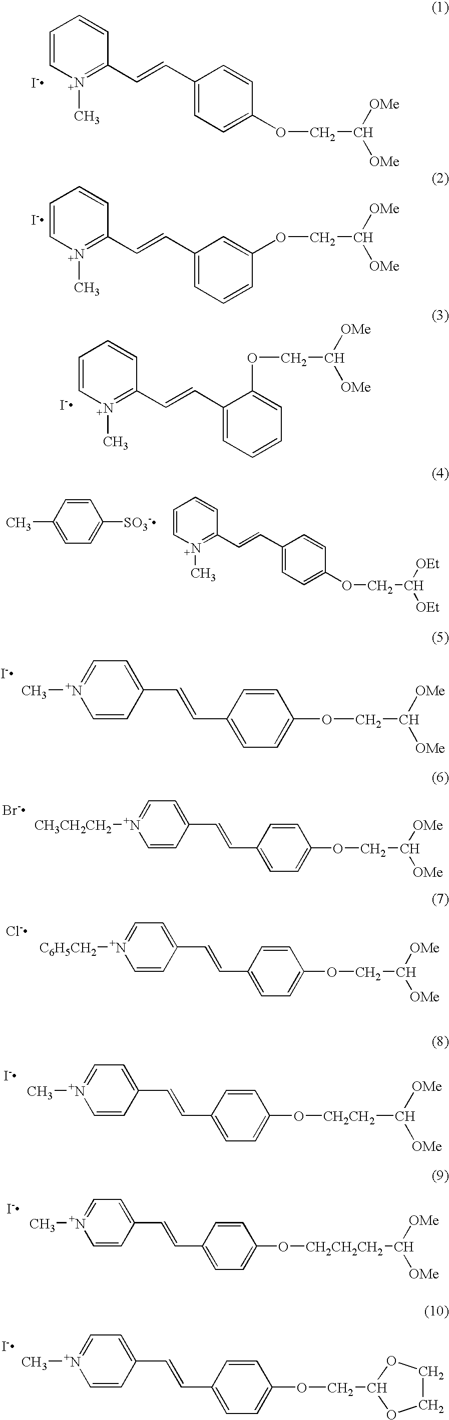

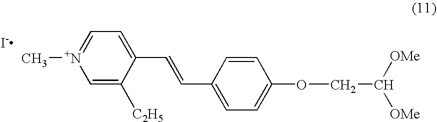

Thus, said second polymeric material is preferably prepared or preparable by providing a compound of general formula

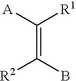

wherein A, B, R¹ and R² are as described above, in an aqueous solvent, (suitably so that molecules of said monomer aggregate) and causing the groups C═C in said compound to react with one another, (for example using UV radiation) to form said second polymeric material.

Said second polymeric material may be of formula

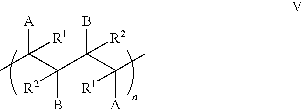

wherein A, B, R¹ and R² are as described above and n is an integer. Integer n is suitably 50 or less, preferably 20 or less, more preferably 10 or less, especially 5 or less. Integer n is suitably at least 1, preferably at least 2, more preferably at least 3.

The ratio of the wt % of said hydrogel (excluding any water encapsulated by said hydrogel) to the wt % of said deliverable material in said delivery means may be at least 10, preferably at least 50, more preferably at least 100. The ratio may be less than 500, preferably less than 250, more preferably less than 200.

Said hydrogel and said deliverable material are preferably intimately mixed with one another. Together they preferably define a substantially homogenous mixture.

Said delivery means preferably comprises water.

Said deliverable material is preferably arranged to diffuse within the delivery means. Said deliverable material may be arranged to diffuse out of the delivery means, in use, for example into a wound bed.

Said delivery means preferably contains at least 2 wt %, preferably at least 25 wt %, more preferably at least 50 wt %, especially at least 80 wt % water. The amount of water may be less than 95 wt %. The level of water may be determined by any suitable means, for example by thermogravimetric analysis.

Said delivery means may include less than 10 wt % of said deliverable material. Suitably said delivery means includes less than 2.5 wt %, preferably less than 1.0 wt %, more preferably less than 0.5 wt %, especially less than 0.2 wt % of said deliverable material. In the most preferred embodiment said delivery means includes less than 0.1 wt % of said deliverable material. Said delivery means may include 100-10000 pm, preferably 100-5000 ppm, more preferably 400-2000 ppm, especially 500-1500 ppm of said deliverable material.

Said delivery means suitably includes less than 30 wt % of organic polymeric materials (for example said hydrophilic polymer and/or said first and/or second polymeric materials and/or a reaction product thereof), preferably less than 20 wt %, more preferably less than 15 wt %, especially less than 12 wt %. The delivery means may include at least 1 wt %, preferably at least 2 wt % of organic polymeric materials. At least some, suitably at least 50 wt %, preferably at least 75 wt %, more preferably at least 90 wt %, especially at least 95 wt % of said organic polymeric material is selected from the group comprising polyvinylalcohol and cross-linked polyvinylalcohol. In said delivery means, the ratio of the sum of wt % of organic polymeric materials to the wt % of said deliverable material may be at least 5, is suitably at least 10, is preferably at least 50 and, more preferably is at least 100. The ratio may be less than 500, preferably less than 250, more preferably less than 200.

A reference to “organic polymeric material,” is suitably not intended to encompass materials which are delivered (i.e. deliverable materials).

Suitably said delivery means comprises:
0.000001 wt % to 5 wt % of a said deliverable material;
2 wt % to 30 wt % of organic polymeric materials; and
65 wt % to 94.999999 wt % of water.

Said delivery means may be in the form of a fluid, for example a viscous fluid or in a solid form, for example in the form of a film or sheet. A viscous fluid may be in the form of an ointment or the like. The physical form of the hydrogel is determined by the wt % of optionally-derivatised, hydrophilic polymer, for example the wt % of hydrophilic polymer and cross-linking means therefor. When the wt % of hydrophilic polymer in the delivery means is about 2 wt % or less then a visco-elastic fluid is formed; when it is greater than about 2 wt %, a rigid gel is formed and the rigidity of the gel is increased as the wt % of optionally-derivatised hydrophilic polymer is increased.

In a preferred embodiment, said delivery means comprises:
0.001 to 1 wt % of a said deliverable material;
2 to 30 wt % of organic polymeric materials; and
69 to 97.999 wt % of water.

When said delivery means is an ointment, the amount of organic polymeric materials may be in the range 2 to 4 wt % with the balance being water, other carriers or excipients. When said delivery means is in solid form (e.g. a film), said delivery means may include 5 to 30 wt %, preferably 10 to 30 wt % of organic polymeric materials.

Suitably, said delivery means comprises:
0.000001 wt % to 5 wt % of a deliverable material;
5 wt % to 30 wt % of polyvinylalcohol and/or cross-linked polyvinylalcohol;
65 wt % to 94.999999 wt % of water.

A deliverable material as described in (ii) may be any protein, protein fragment, peptide or amino acid. Said deliverable material preferably includes one or more amide bonds. It may be comprise a single protein, rather than a mixture. A deliverable material as described in (ii) is preferably naturally-occurring or is an analogue of a naturally-occurring material. It is more preferably naturally-occurring. Whilst the material described in (ii) is preferably of a type which is naturally-occurring, it may be a synthetic version of such a naturally-occurring material or a modification thereof. Said material described in (ii) may be a recombinant isoform of a material or may comprise a modified material produced by recombinant techniques.

Said material described in (ii) may be a recombinant form of one or more materials secreted or excreted from the organism Lucilia sericata or Drosophila melanogaster.

A deliverable material described in (ii) may comprise an extract from a material of natural origin, for example from an animal or plant, preferably from an animal. It preferably comprises an extract from an insect, preferably when in its larval stage. Such an extract may optionally be purified and/or derivatised to produce a said deliverable material.

Said deliverable material described may be of a type which is excreted/secreted by the organism Lucilia sericata. It may comprise an isolated protein. Such a protein may exhibit optimum proteolytic activity against FITC-casein at a pH of 8.0 to 8.5; it may exhibit proteolytic activity against Tosyl-Gly-Pro-Arg-AMC but not against Suc-Ala-Ala-Phe-AMC; its proteolytic activity against FITC-casein and Tosyl-Gly-Pro-Arg-AMC may be inhibited by the serine proteinase inhibitors PMSF and APMSF; and/or it may be bound by immobilised aminobenzamidine. It may have each of the aforementioned features. Said protein may have a molecular weight of approximately 25 kDa.

Said deliverable material described in (ii) may comprise one or more peptides selected from the group consisting of
Ser-Phe-Leu-Leu-Arg-Asn;
Ser-Leu-Ile-Gly-Lys-Val;
Thr-Phe-Arg-Gly-Ala-Pro;
Gly-Tyr-Pro-Gly-Gln-Val, and
   a peptide having an N-terminal sequence selected from:
Ser-Phe-Leu-Leu-Arg-Asn;
Ser-Leu-Ile-Gly-Lys-Val;
Thr-Phe-Arg-Gly-Ala-Pro; or
Gly-Tyr-Pro-Gly-Gln-Val,
   or a protected analogue therefor which is protected against aminopeptidase activity.

A deliverable material as described may be as described in WO01/31033, the contents of which are incorporated herein by reference.

Said deliverable material described may comprise a substance having N-acyl homoserine lactone degradant activity obtained from the secretions/excretions of Lucilia sericata. Said deliverable material may comprise a serine proteinase, or a glycosidase or a substance having cecropin-like activity, each preferably being isolated from secretions/excretions obtained from Lucilia sericata or analogues thereof. Such materials may be as described in WO03/075654, the contents of which are incorporated herein by reference.

Said deliverable material described may comprise a toll receptor ligand, or a precursor thereof, which ligand may be a member of the cysteine knot superfamily of proteins. The ligand is preferably an insect-derived protein or an active portion or analogue thereof and is suitably derived from Drosophila melanogaster or Lucilia sericata. The active portion of the protein may comprise a C-terminal 106 amino acid peptide. It may be as described in WO03/043669, the contents of which are incorporated herein by reference.

A deliverable material described in (iii) is preferably derived from larvae of the organisms described. The secretion/excretion may be used substantially whole or it may be purified and/or fractions of the material may be isolated.

Preferably, a said secretion/excretion described herein is from the organism Lucilia sericata.

Said delivery means may include an additional deliverable material. Such a material may have anti-bacterial properties, for example it may be an antibiotic, for example a tetracycline antibiotic; it may comprise silver; it may be a molecule which interrupts signalling in bacterial colonies (e.g. it may be biofilm), fungi, mould, mycoplasma; it may have bacteriostatic properties. When an additional material is included, the ratio of the wt % of said deliverable material to the wt % of said additional deliverable material may be in the range 0.1 to 10, preferably 0.2 to 5.

Said delivery means is preferably sterile

Said delivery means has preferably been sterilised by heat, electron beam radiation or gamma radiation.

According to a second aspect of the invention, there is provided a method of manufacturing a delivery means according to the first aspect, the method comprising contacting a deliverable material according to said first aspect with an hydrogel or with precursor material arranged to form an hydrogel.

In a first embodiment, a hydrogel which includes less than the maximum level of water which may be encapsulated therein or a precursor material which comprises a dehydrated hydrogel may be contacted with a formulation comprising said deliverable material suitably so that said deliverable material becomes absorbed into the hydrogel or precursor material. When the precursor material is contacted as described, it suitably forms a hydrogel. The deliverable material may be contacted with said hydrogel or precursor material when the level of water encapsulated in the hydrogel or precursor material is less than the maximum level of water which may be encapsulated therein. The ratio of the wt % of the maximum level of water which can be contained in the hydrogel or precursor material to the wt % in the hydrogel or precursor material when contacted with said deliverable material is preferably greater than 2, more preferably greater than 10. Said hydrogel may be substantially dehydrated when initially contacted with said deliverable material and/or said precursor material may comprise a substantially fully dehydrated hydrogel.

Said formulation comprising said deliverable material preferably comprises an aqueous formulation of said deliverable material. Said formulation may include greater than 90 wt %, preferably greater than 95 wt %, more preferably greater than 98 wt %, especially greater than 99 wt % water. Said formulation may include less than 2 wt %, preferably less than 1.5 wt %, more preferably less than 1 wt %, especially less than 0.5 wt %, most preferably less than 0.15 wt % of said deliverable material. The amount of deliverable material may be at least 0.005 wt %, preferably at least 0.001 wt %, more preferably at least 0.05 wt %.

Advantageously, formation of said delivery means as described does not require the deliverable material to be subjected to harsh conditions, for example of temperature or pH.

Suitably, the method involves contacting the hydrogel or precursor material with an aqueous formulation comprising 0.1 to 10 mg/ml, preferably 0.25 to 5 mg/ml, more preferably 0.5 to 2.5 mg/ml of said deliverable material. Contact may be carried out at a temperature in the range 5 to 50° C., preferably 10 to 35° C., especially at ambient temperature.

The hydrogel or precursor material may be prepared by dehydrating a hydrogel, for example a hydrogel as described herein or as prepared according to the second embodiment below, but excluding the incorporation of the deliverable material with the hydrophilic polymer and cross-linking means.

In a second embodiment, said deliverable material may be contacted with precursor material arranged to form said hydrogel and suitably to encapsulate said deliverable material. For example, said precursor material may comprise a hydrophilic polymer as described according to the first aspect and a cross-linking means. The aforesaid may be contacted with said deliverable material, suitably in the presence of a catalyst for catalysing a reaction between the hydrophilic polymer and cross-linking means. The ratio of the weight of said hydrophilic polymer to the weight of cross-linking means used in the method is preferably in the range 10 to 100. Suitably, the ratio may be at least 15, preferably at least 20, more preferably at least 30. The ratio may be less than 90, preferably less than 80, more preferably less than 70.

The level of organic materials used to form the hydrogel may be adjusted to vary the physical properties of the hydrogel. For example, the sum of the wt % of hydrophilic polymer and cross-linking means may be in the range 0.5 wt % to 20 wt %, preferably in the range 1 wt % to 15 wt %. At the lower end of the range, the hydrogel may be visco-elastic and may be suitable for incorporation into an ointment for topical application or for impregnation into a porous material. At the higher end of the range, a solid gel forms which may itself be used as a layer of a dressing in use.

In the second embodiment, the contact of said deliverable material with precursor material may be carried out in the presence of up to 99.5 wt % water. The level of water may be 85 to 95 wt %. The second embodiment may involve contact of 0.5 to 20 wt % of organic material selected from hydrophilic polymer and cross-linking means, 79.99 to 99.49 wt % of water and at least 0.01 wt % of deliverable material.

Said method of the second aspect may comprise the step of sterilising the hydrogel, for example using heat, electron beam radiation or gamma radiation.

According to a third aspect of the invention, there is provided a treatment material, suitably for a wound, lesion or other area of a human or animal body which requires treatment, said treatment material comprising a delivery means according to the first aspect and/or made as described according to said second aspect.

Said treatment material may comprise a dressing wherein said delivery means is impregnated in a material; or said delivery means could be provided in the form of a sheet or film and/or a rigid material; or said delivery means could be in the form of a fluid (e.g. ointment).

The deliverable material may be incorporated to the level described according to the first aspect and/or as described according to the second aspect.

The treatment material is preferably provided in a substantially sterile environment (e.g. package or receptacle such as a tube) prior to use.

Said treatment material is preferably a dressing.

When said material is a dressing, said dressing may comprise a first face via which deliverable material is arranged to pass to contact a part of the human or animal body which requires treatment and, upstream of said first face, said dressing includes an impermeable barrier which substantially resists passage of fluid (e.g. water) therethrough. Thus, said dressing is preferably arranged for passage of fluid therefrom in substantially a single direction.

According to a fourth aspect of the invention, there is provided a method of treating a wound, lesion or other area of a human or animal body which requires treatment, the method comprising contacting an area to be treated with a delivery means according to the first aspect and/or with a treatment material according to the third aspect.

According to a fifth aspect of the invention, there is provided the use of a delivery means of the first aspect for the manufacture of a material, for example a dressing or formulation for topical application, for treatment of a wound, lesion or other area of a human body which requires treatment.

Preferably, in accordance with the inventions of the fourth and fifth aspects, the wound, lesion or other area may be treatment after debridement of the wound, lesion or other area has taken place. Thus, treatment of the wound in accordance with the fourth and fifth aspects may include a first step which comprises causing debridement of the wound, lesion or other area, suitably using a material other than one comprising a delivery means as described herein; and a second step, after the first step, which comprises use of the delivery means or treatment material as described herein. The first step may comprise the use of maggots. The second step preferably involves promoting healing of the wound, lesion or other area using a said delivery means and/or treatment material as described herein.

Any feature of any aspect of any invention or embodiment described herein may be combined with any feature of any aspect of any other invention or embodiment described herein mutatis mutandis.

Specific embodiments of the invention will now be described by way of example, with reference to the accompanying figures in which:

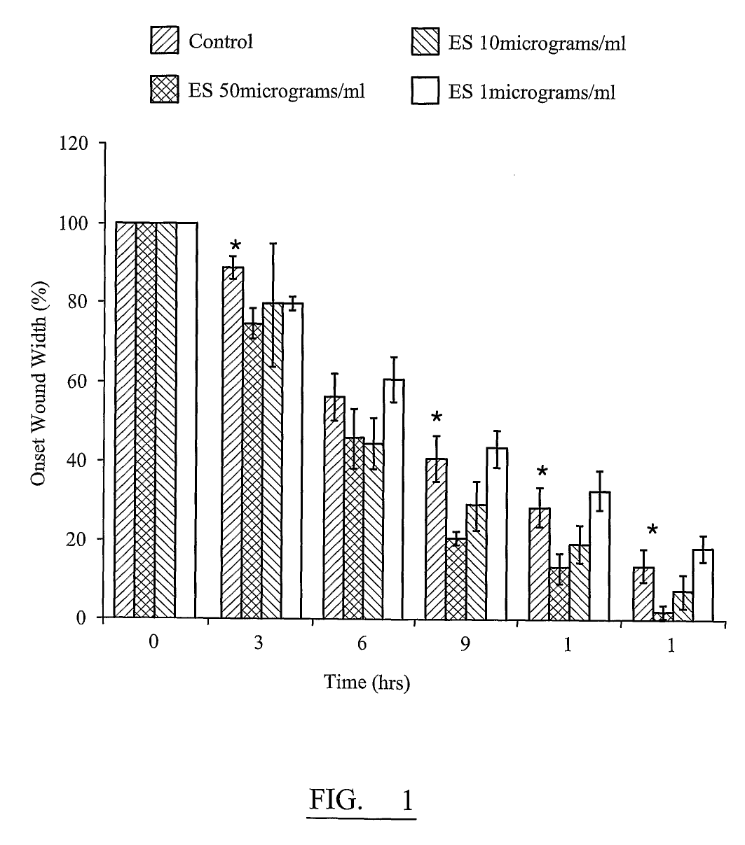

FIG. 1 illustrates the close response relationship between concentration of active material (ES) in the culture media and the ratio of wound closure;

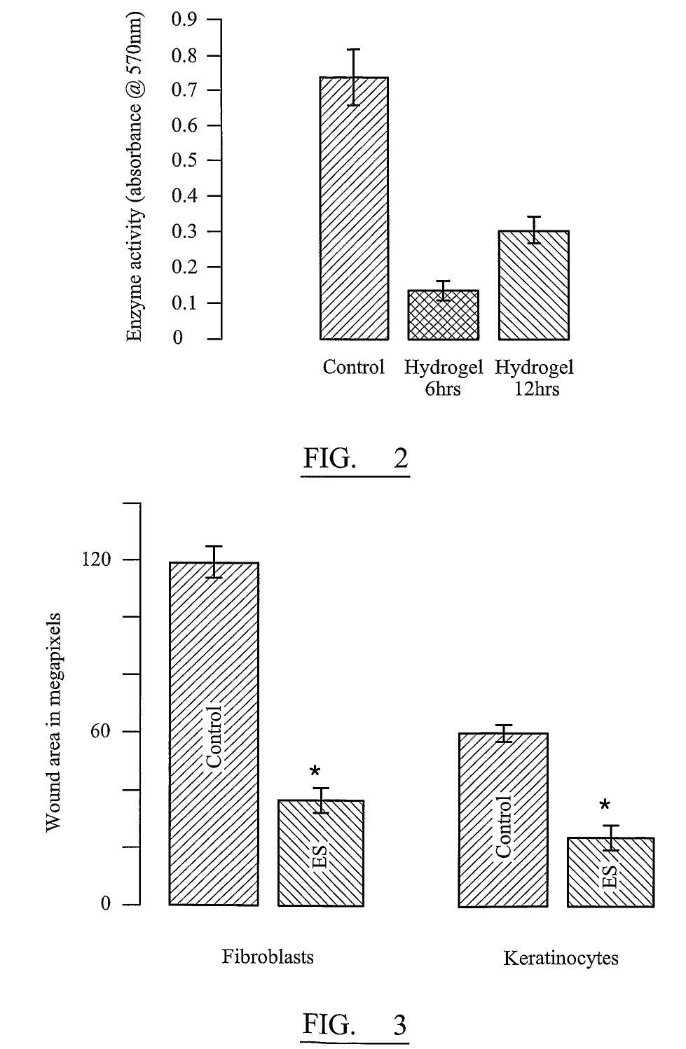

FIG. 2 summarises the results of a colorimetric protease assay for estimation of active material (ES) release from a hydrogel material;

FIG. 3 summarises the results of studies on a model wound growth medium.

The following material is referred to hereinafter:

Poval 220—a polyvinylalcohol obtained from Kuraray having a viscosity, measured on a 4% aqueous solution at 20° C. (determined by a Brookfield synchronised-meter rotary-type viscometer), of 30.mPa·s and a degree of hydrolysis (saponification) of about 88% mol %. The molecular weight is about 130,000.

### EXAMPLE 1

### Preparation of Larval Excretory/Secretory Products (ES)

ES was extracted in phosphate buffered saline (0.01M PBS, pH 7.3) from sterile L. sericata larvae (LarvE, Surgical Materials testing laboratory, Cardiff, UK) shortly after hatching, according to the method described by Horobin et al (Horobin A. J, Shakesheff K. M, Woodrow S, Robinson C, Pritchard D. I. (2003) Maggots and wound healing: The Effects of Lucilia sericata Larval Secretions upon Human Dermal Fibroblasts. British Journal of Dermatology 148: 923-33). ES solution was subsequently freeze-dried for storage and then reconstituted in sterile water for experimental use at a protein concentration equivalent to 50 μg/ml for migration studies and 1 mg/ml for the release studies. Larval ES has been shown to be highly stable at temperatures of less than 65° C., and activity assay confirmed that no deleterious effect of incubation temperature of 37° C. on ES levels in media was observed.

### EXAMPLE 2

### Preparation of poly(1,4-di(4-(N-methylpyridinyl))-2,3-di(4-(1-formylphenyl)butylidene

This was prepared as described in Example 1 of PCT/GB97/02529, the contents of which are incorporated herein by reference. In the method, an aqueous solution of greater than 1 wt % of 4-(4-formylphenylethenyl)-1-methylpyridinium methosulphonate (SbQ) was prepared by mixing the SbQ with water at ambient temperature. Under such conditions, the SbQ molecules form aggregates. The solution was then exposed to ultraviolet light. This results in a photochemical reaction between the carbon-carbon double bonds of adjacent 4-(4-formylphenylethenyl)-1-methylpyridinium methosulphate molecules (I) in the aggregate, producing a polymer, poly(1,4-di(4-(N-methylpyridinyl))-2,3-di(4-(1-formylphenyl)butylidene methosulphonate (II), as shown in the reaction scheme below. It should be appreciated that the anions of compounds I and II have been omitted in the interests of clarity.

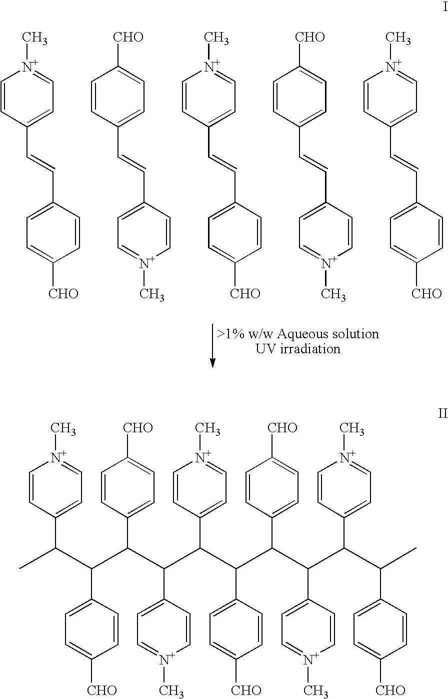

### EXAMPLE 3

### Preparation of Hydrogel

A 10% w/w solution of 88% hydrolysed poly(vinylalcohol) of molecular weight 300,000 was prepared and to this was added 1% w/w of the butylidene polymer of Example 2. The mixture was degassed under vacuum and polymerised by gentle addition of 20% w/w hydrochloric acid to a final concentration of 0.02 wt % taking care to avoid introduction of air bubbles. The mixture was quickly poured into non-adherent plastic dishes to produce a gel sheet of final depth of 1 mm. After 12 hours, the polymerised gel was gently detached and washed with RO water until the gel surface gave a pH reading of 6-7 (tested with a flat-bottom pH electrode; Hanna). Discs of 5 mm diameter were punched from the gel sheet (designed to fit into the wells of a standard ELISA plate when hydrated). Discs were thoroughly dehydrated by drying in a vacuum oven at 60° C.

### EXAMPLE 4

### First Method for Incorporation of ES into Hydrogel

The dehydrated discs of Example 3 were rehydrated in 1 mg/ml ES prepared in 0.01M phosphate buffered saline (PBS), pH 7.5 for 12 hours. The concentration of ES was selected with prior knowledge of the capacity for liquid uptake of the discs and designed to give maximum release of 50 μg/ml ES into the cell cultures. Control discs were rehydrated with PBS alone. Discs were then rinsed with PBS to remove exogenous solution and blotted dry. Discs were stored at 4° C. prior to use.

Discs comprising other concentrations of ES were also prepared in a similar manner.

### EXAMPLE 5

### Assessment of ES-Containing Hydrogel Discs

(i) Release of ES from Hydrogel Discs

This was confirmed by suspension of hydrogel discs impregnated with ES (1 mg/ml) and untreated discs in Tris buffered saline, pH 8 (TBS) for 6 hours and 12 hours. For colour comparison, a reference solution of ES 50 μg/ml was also prepared in TBS. Aliquots of supernatant were then aspirated at 6 hours and at 12 hours after disc submersion and protease activity assayed by addition of 10% v/v Protease Substrate Cocktail (Protease Substrate Cocktail I, Calbiochem) prepared in TBS. This agent comprised a broad spectrum of specific calorimetric substrates for serine, cysteine, aspartic and aminopeptidase proteases.

In order to submerge discs in the model wound growth media with minimal mechanical disturbance to the delicate cell monolayer cell, hydrogel discs impregnated with ES and control discs were suspended within the supernatant media approximately 1 mm above the cell cultures by means of Costar Netwell® well inserts (Corning, UK) enabling release of substances from hydrogel discs by diffusion through a 500 μm polyester mesh insert bottom, directly onto growth media.

(ii) Immunohisto and Immunocytochemical Detection of pTyr Expression in Cell Cultures.

Localisation of phosphorylated tyrosine (pTyr) was carried out in adherent model wound cultures of 3T3 fibroblasts following incubation for 12 hours (as described above) with (a) ES 50 μg/ml (b) control (c) ES 50 μg/ml and Protease Inhibitor Cocktail (PIC; Sigma) diluted 1/200 in media. Cells were fixed for 10 minutes by addition of ice-cold methanol:acetic acid in ratio 50:50 to the growth media. Following aspiration and rinsing with wash solution (PBS/0.05% Tween 20), cell cultures were incubated with blocking agent (6% non-fat dry skimmed milk powder in PBS/0.01% Tween 20) for 3 hours at 21° C. Following aspiration and rinse with wash solution cultures were incubated for 3 hours at 21° C. with monoclonal mouse anti-phosphotyrosine-peroxidase conjugate (mAb pTyr-HRP; clone PT-66; Sigma Aldrich, Poole, Dorset) at a concentration of 1:10,000 mAb pTyr in antibody diluent (1% non-fat dry skimmed milk powder in PBS/0.01% Tween 20). Following aspiration and several rinses with wash solution, cultures were incubated with a proprietary tetramethylbenzidine (TMB) peroxidase substrate (TMB liquid substrate system for membranes, Sigma Aldrich). This form of TMB produces an insoluble blue reaction product that precipitates in situ as a marker of mAb pTyr-HRP localization. After allowing 15 minutes for maximal TMB colour development, excess TMB solution was aspirated and cultures rinsed with wash solution. Cultures were maintained in PBS for microscopy.

Quantification of phosphorylated tyrosine (pTyr) expression was carried out in model wound cultures of 3T3 fibroblasts following incubation with ES 50 μg/ml or control for 12 hours (as described above). Cell monolayers were stripped from the well culture surface with a cell-scraper and cells/growth media aspirated and pelleted by low-speed centrifugation (2500 g, 5 minutes). The supernatant was discarded and cells resuspended in 200 μl of ice-cold Phosphosafe™ Extraction reagent (Calbiochem) and incubated for 15 minutes at 21° C. Cell suspensions were pelleted by centrifugation at 15,000 g for 5 minutes. Supernatant was transferred to a 96-well multiwell plate and incubated for 2 hours at 21° C. Wells were then rinsed with wash solution and incubated with blocking agent (6% non-fat dry skimmed milk powder in PBS/0.01% Tween 20) for 3 hours at 21° C. Following aspiration and rinse with wash solution, wells were incubated for 3 hours at 21° C. with monoclonal mouse anti-phosphotyrosine-peroxidase conjugate (mAb pTyr; clone PT-66; Sigma Aldrich, Poole, Dorset) at a concentration of 1:60,000 mAb pTyr in antibody diluent (1% non-fat dry skimmed milk powder in PBS/0.01% Tween 20). Following aspiration and several rinses with wash solution, wells were incubated with a proprietary tetramethylbenzidine (TMB) peroxidase substrate (TMB liquid substrate system for ELISA, Sigma Aldrich). After allowing 15 minutes for maximal TMB colour development, absorbance readings were measured at 655 nm using a spectrophotometric multi-well plate reader (Biorad).

(iii) Results

There was a significant dose-response relationship between the concentration of ES in the culture media and the rate of wound closure in 3T3 fibroblast monolayer cultures, with 50 μg/ml producing the most rapid closure (FIG. 1). More detailed analysis of wound surface area in 3T3 fibroblast monolayer cultures after 12 hours in liquid media supplemented with maggot extract at 50 μg/ml confirmed the markedly enhanced rate of closure.

Colorimetric protease assay for estimation of ES release from the hydrogel material showed a time dependant increase in ES in the disc supernatant (FIG. 2). Studies of the effect of ES released from the hydrogel material into the 3T3 fibroblasts and HaCaT model wound growth medium showed a significant (p<0.001 for both cell types) increases in rate of model wound closure following incubation with ES impregnated discs for 12 hours (FIG. 3).

### EXAMPLE 6

### Second Method for Preparation of ES into Hydrogel

An aqueous solution comprising 10 wt % Poval 220 polyvinylalcohol and 0.5 wt % of the butylidene polymer of Example 2 was prepared. A typical method for its preparation comprises dissolving the powderous Poval polyvinylalcohol slowly and with constant stirring in a solution of the butylidene polymer. Complete dissolution may be achieved by maintaining the solution at a temperature of 60° C. for a period of 6 hours. To the solution prepared was added the ES solution with mixing.

The mixture was then acidified to pH 2.5. The acidified mixture was poured into 100 mm diameter Petri-dishes to a depth of 3 mm and allowed to gel for 48 hours. As a result a thin film of gel incorporating ES is formed.

### EXAMPLE 7

### Sterilisation of the Gel of Example 6

The films of Examples 4 and 6 can be sterilised using known methods.

The hydrogel incorporating ES may be incorporated into a wound dressing. For example it may comprise a layer of a dressing which contacts a wound in use or it may define an inner layer of a dressing which is separated from a wound in use by one or more other layers. In another embodiment, a hydrogel may be incorporated into a porous carrier. Referring to Example 6, rather than the formulation being poured into a dish and a film formed, the formulation may be used to impregnate a porous material for example a fabric which may act as a carrier. In a further embodiment, the formulation may be used to prepare a material, for example an ointment, for topical application to a wound. In this case, a mixture of polyvinylalcohol, butylidene polymer and acid may be mixed with a carrier arranged to define a cream.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A delivery means for delivering a deliverable material, said delivery means comprising a hydrogel and said deliverable material comprising:
(i) a decomposable material which in the absence of said hydrogel decomposes and/or is permanently denatured if sterilised using heat, electron beam radiation or gamma radiation;
(ii) a protein, protein fragment, peptide or amino acid; or
(iii) a secretion or excretion from the organism Lucilia sericata or Drosophila melanogaster.

2. A delivery means according to claim 1, wherein said decomposable material is such that, in the absence of said hydrogel, it will decompose and/or be permanently denatured if subjected to a temperature of greater than 100° C.; gamma radiation of at least 40 kV for at least 1 minute; or electron beam radiation of at least 18 kV for at least 1 minute.

3. A delivery means according to claim 1, wherein said hydrogel comprises an optionally derivatised hydrophilic polymer.

4. A delivery means according to claim 3, wherein said hydrophilic polymer is selected from polymethacrylic acid polymers, polyimides, polyvinylalcohol and copolymers of the aforesaid.

5. A delivery means according to claim 1, wherein said hydrogel comprises an optionally derivatised polyvinylalcohol.

6. A delivery means according to claim 1, wherein said hydrogel comprises an optionally derivatised polyvinylalcohol which consists essentially of vinyl alcohol and vinyl acetate functional groups, wherein the polyvinylalcohol is hydrolysed to an extent of at least 25 mole % and less than 95 mole %.

7. A delivery means according to claim 1, wherein said hydrogel comprises a hydrophilic polymer which is cross-linked by a cross-linking means.

8. A delivery means according to claim 1, wherein said hydrogel includes a material formed by cross-linking polyvinylalcohol using a cross-linking material having at least two aldehyde groups.

9. A delivery means according to claim 8, wherein said cross-linking material comprises a polymeric material which includes a repeat unit of formula 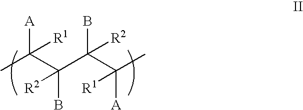
wherein A and B are the same or different, are selected from optionally-substituted aromatic and heteroaromatic groups and at least one comprises a relatively polar atom or group and R¹ and R² independently comprise relatively non-polar atoms or groups.

10. A delivery means according to claim 1, wherein the ratio of the wt % of said hydrogel to the wt % of said deliverable material is at least 10 and said ratio is less than 500.

11. A delivery means according to claim 1, wherein said hydrogel and said deliverable material are intimately mixed with one another and define a substantially homogenous mixture.

12. A delivery means according to claim 1, wherein said delivery means includes at least 50 wt % and less than 95 wt % of water and includes less than 10 wt % of said deliverable material.

13. A delivery means according to claim 1, which comprises:
0.000001 wt % to 5 wt % of a said deliverable material;
2 wt % to 30 wt % of organic polymeric materials; and
65 wt % to 94.999999 wt % of water.

14. A method of manufacturing a delivery means according to claim 1, the method comprising contacting a deliverable material according to any preceding claim with an hydrogel or with precursor material arranged to form a hydrogel.

15. A method according to claim 14, wherein a hydrogel which includes less than the maximum level of water which may be encapsulated therein or a precursor material which comprises a dehydrated hydrogel is contacted with a formulation comprising said deliverable material so that said deliverable material becomes absorbed into the hydrogel or precursor material.

16. A method according to claim 14, wherein the ratio of the wt % of the maximum level of water which can be contained in the hydrogel or precursor material to the wt % in the hydrogel or precursor material when contacted with said deliverable material is greater than 2.

17. A method according to claim 14, which involves contacting the hydrogel or precursor material with an aqueous formulation comprising 0.1 to 10 mg/ml of said deliverable material at a temperature in the range 5 to 50° C.

18. A method according to claim 14, wherein said deliverable material is contacted with precursor material arranged to form said hydrogel and to encapsulate said deliverable material.

19. A method according to claim 14, which includes the step of sterilising the hydrogel.

20. A treatment material comprising a delivery means according to claim 1.

21. A method of treating a wound, a lesion or other area of a human or animal body which requires treatment, the method comprising contacting an area to be treated with a delivery means according to claim 1.

22. (canceled)

23. A delivery means for delivering a deliverable material to a wound bed, said delivery means comprising a hydrogel and a deliverable material which are intimately mixed with one another and define a substantially homogenous mixture, wherein said deliverable material comprises:
(i) a decomposable material which in the absence of said hydrogel decomposes and/or is permanently denatured if sterilised using heat, electron beam radiation or gamma radiation;
(ii) a protein, protein fragment, peptide or amino acid; or
(iii) a secretion or excretion from the organism Lucilia sericata or Drosophila melanogaster,
wherein said hydrogel comprises an optionally derivatised polyvinylalcohol which consists essentially of vinyl alcohol and vinyl acetate functional groups, wherein the polyvinylalcohol is hydrolysed to an extent of at least 25 mole % and less than 95 mole %; and
wherein said delivery means comprises:
0.000001 wt % to 5 wt % of said deliverable material;
2 wt % to 30 wt % of organic polymeric materials; and
65 wt % to 94.999999 wt % of water.
